(19) United States
(12) Patent Application Publication
(10) Pub. No.: US 2024/0290422 A1
(43) Pub. Date: Aug. 29, 2024

(54) METHODS FOR IDENTIFYING MUTATIONS USING MACHINE LEARNING

(71) Applicant: STRATA ONCOLOGY, INC., Ann Arbor, MI (US)

(72) Inventors: Scott Arthur Tomlins, Ann Arbor, MI (US); Daniel Reed Rhodes, Chelsea, MI (US); David Bryan Johnson, Chelsea, MI (US)

(21) Appl. No.: 18/571,652

(22) PCT Filed: Jun. 17, 2022
(86) PCT No.: PCT/US2022/034115

§ 371 Date: Dec. 18, 2023

Related U.S. Application Data
(60) Provisional application No. 63/211,891, filed on Jun. 17, 2021.

(51) Int. Cl. G16B 20/00; G16B 40/20

(52) U.S. Cl. CPC G16B 20/00; G16B 40/20

(57) ABSTRACT

Disclosed herein are methods for identifying mutations from a patient sample, by evaluating, using a computer having a machine learning classifier, a candidate variant against a plurality of decision trees trained to detect mutations in the candidate variant with a gradient boosting algorithm.

### RELATED APPLICATION

This application claims priority to, and the benefit of, co-pending U.S. Provisional No. 63/211,891, filed Jun. 17, 2021. The disclosure of said provisional application is hereby incorporated by reference in its entirety.

### BACKGROUND OF THE INVENTION

Next Generation Sequencing (NGS)-based cancer tests (e.g., variant calling) are commonly used in the advanced cancer setting to inform treatment selection of targeted therapy (e.g., due to alterations in ALK, EGFR or BRAF), and for research. While multiple NGS-based companion diagnostic (CDx) cancer tests have now been FDA approved, their uptake has been limited by: (1) single-site approvals (e.g., FoundationOne CDx™ [F1 CDx]) that compete with regional laboratories who wish to better serve their local patient populations, (2) impractical sample input requirements for capture-based NGS IVD tests (e.g., F1 CDx requires tumor surface area >25 mm²) and (3) insufficient content in kitted products (e.g., Oncomine Target Dx, Praxis Extended Ras Panel), which took many years to develop, but when launched, no longer met current medical needs given the ever expanding list of biomarkers recommended or required by professional organizations and payors.

Hence, the majority of molecular testing for patients with advanced cancer in the U.S. continues to occur in CLIA-certified laboratories as custom single-gene or NGS laboratory-developed tests (LDTs). In the 2018 B Mulitgene Tumor Panel CAP proficiency test, of 97, 101 and 101 laboratories reporting testing for EGFR, BRAF and KRAS mutations, respectively, only 23 (24%), 7 (7%) and 2 (2%) used FDA cleared or approved companion diagnostic tests. While LDTs can be developed more quickly to meet changing medical needs, they are at increased risk of producing inaccurate or inconsistent results due to variability in laboratory methods, bioinformatics and data interpretation, and in some cases, inadequate analytical and clinical validation for their intended use. Hence, as NGS tests become more complex, this risk has the potential to increase.

US 2019/0189242 A1 (Published Jun. 20, 2019), provides a variant calling method utilizing machine learning. However, as detailed in paragraph [0166], machine learning was accomplished using an artificially created “spike-in” training set. Thus, the method of US 2019/0189242 A1 has degraded performance when calling “real world” samples that often comprise low quality samples.

Thus, there remains a need in the art for standardized and better variant calling methods that can be applied by any laboratory analyzing NGS sequences.

### SUMMARY OF THE INVENTION

Some aspects of the present disclosure are directed to a method for identifying mutations from a patient sample, comprising: evaluating, using a computer having a machine learning classifier, a candidate variant against a plurality of decision trees trained to detect mutations in the candidate variant with a gradient boosting algorithm, wherein each decision tree classifies the candidate variant as at least one of present or not present; and classifying, using the computer, the candidate variant as a real mutation based on classifications of each of the plurality of decision trees, wherein the decision trees receive the following parameters: min_AF_ratio_50; min_alt_count; min_AF_softclip_ratio_99; count; and within_tumor_prior.

In some embodiments, the decision trees also receive the following parameters: min_AF_ratio_100, min_AF_softclip_ratio_100, min_AF_softclip, min_AF_softclip_ratio_90, and max_alt_count.

In some embodiments, the decision trees also receive one or more of the following parameters: max_AF_ratio_100, AF, frac_amps_with_evidence, pan_tumor_prior, amplicon_variant_count, AF_frac_pos_max, analysis_variant_count, min_depth, min_AF, pct_noisy_positions, avg_cov_rel_chip, max_AF_ratio_99, AF_softclip, pan_tumor_prior_gene, min_AF_ratio_99, within_tumor_prior_gene, max_AF_ratio_50, min_AF_softclip_ratio_50, max_AF_ratio_90, depth, num_covering_amps, DNA_avg_coverage, max_AF_softclip, frac_max_af, max_depth, max_AF_softclip_ratio_99, min_AF_ratio_90, max_AF, max_AF_softclip_ratio_100, max_AF_softclip_ratio_90, max_softclip_count, best_strand_bias, max_AF_softclip_ratio_50, and min_softclip_count.

In some embodiments, evaluating the candidate variant further comprises evaluating the candidate variant using a random forest classifier. In some embodiments, the plurality of decision trees further comprises at least one thousand decision trees. In some embodiments, the plurality of decision trees further comprises a plurality of decision trees for each mutation.

In some embodiments, the method further comprises training the machine learning classifier using a training data set of sequences that include identified mutations. In some embodiments, the training data set of sequences was obtained in part from low quality biological samples and wherein the mutations are identified via expert review.

In some embodiments, training the machine learning classifier further comprises optimizing parameters of the machine learning classifier until the machine learning classifier produces output describing the known mutations. In some embodiments, optimizing parameters of the machine learning classifier further comprises selecting a plurality of feature categories. In some embodiments, the machine learning classifier is selected from the group consisting of: a neural network, Bayesian classifier, logistic regression, decision tree, gradient-boosted tree, multilayer perceptron, one-vs-rest, and Naive Bayes.

In some embodiments, the method further comprises generating, using the computer, an overall confidence score for the candidate variant being a real mutation based on the classifications of all of the plurality of decision trees. In some embodiments, the method further comprises providing a report that describes the candidate variant as including the mutation.

In some embodiments, one or more of the decision trees receive parameters selected from the group consisting of: sample type; FASTQ quality score; alignment score; read coverage; and an estimated probability of error. In some embodiments, the training data set comprises a plurality of known single-nucleotide variants (SNVs) and insertions/deletions (indels), the method comprising: detecting at least one mutation in the nucleic acid; validating the detected mutation as present in the nucleic acid using the classification model; and providing a report that describes the nucleic as including the mutation.

In some embodiments, a decision tree classifies the candidate variant as present or absent (e.g., likely noise or other sequencing artifact). In some embodiments, the decision tree does not classify whether or not the candidate variant is germline.

Some aspects of the present disclosure are directed to a system for identifying mutations from a patient sample, comprising: a computer system having a processor, memory and a plurality of lines of instructions; a machine learning classifier executed by the processor of the computer system, the machine learning classifier being configured to: evaluate a candidate variant against the plurality of decision trees trained to detect mutations in the candidate variant, wherein each decision tree classifies the candidate variant as present or absent (e.g., likely noise or other sequencing artifact), based on classifications of each of the plurality of decision trees.

In some embodiments, the machine learning classifier is further configured to evaluate the candidate variant using a random forest classifier. In some embodiments, the plurality of decision trees further comprises at least one thousand decision trees. In some embodiments, the plurality of decision trees further comprises a plurality of decision trees for each mutation. In some embodiments, the processor is further configured to train the machine learning classifier using a training data set of sequences that include known mutations. In some embodiments, the training data set of sequences was obtained in part from low quality biological samples and wherein the mutations are identified via expert review. In some embodiments, the processor is further configured to optimize parameters of the machine learning classifier until the machine learning classifier produces output describing the known mutations. In some embodiments, the processor is further configured to select a plurality of feature categories. In some embodiments, the machine learning classifier is selected from the group consisting of: a neural network, Bayesian classifier, logistic regression, decision tree, gradient-boosted tree, multilayer perceptron, one-vs-rest, and Naive Bayes. In some embodiments, the processor is further configured to generate an overall confidence score for the candidate variant being a real mutation (vs. sequencing artifact) based on the classifications of all of the plurality of decision trees.

In some embodiments of the methods and systems described herein, the sample is from plasma, blood, serum, saliva, sputum, stool, a tumor, cell free DNA, circulating tumor cell, or other biological sample. In some embodiments, the sample is from a subject having or at risk of having cancer. In some embodiments, the cancer is selected from lung, bladder, colon, gastric, head and neck, breast, prostate, non-small cell lung adenocarcinoma, non-small cell lung squamous cell carcinoma, bladder urothelial carcinoma, colorectal, brain or pancreatic cancer.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows sequence read information for KRAS chr12:25398280 C>T.

FIG. 2 shows sequence read information for BRCA1 chr17:41256098 A>G.

FIG. 3 shows sequence read information for MSH6 chr2:48010515 G>A.

### DETAILED DESCRIPTION OF THE INVENTION

Some aspects of the present disclosure are directed to a method for identifying mutations from a patient sample, comprising: evaluating, using a computer having a machine learning classifier, a candidate variant against a plurality of decision trees trained to detect mutations in the candidate variant with a gradient boosting algorithm, wherein each decision tree classifies the candidate variant as at least one of present or not present; and classifying, using the computer, the candidate variant as a real mutation based on classifications of each of the plurality of decision trees, wherein the decision trees receive the following parameters: min_AF_ratio_50, min_alt_count, min_AF_softclip_ratio_99, count, and within_tumor_prior.

In some embodiments, the decision trees also receive one or more of the following parameters: min_AF_ratio_100, min_AF_softclip_ratio_100, min_AF_softclip, min_AF_softclip_ratio_90, and max_alt_count. In some embodiments, the decision trees also receive two or more of the following parameters: min_AF_ratio_100, min_AF_softclip_ratio_100, min_AF_softclip, min_AF_softclip_ratio_90, and max_alt_count. In some embodiments, the decision trees also receive three or more of the following parameters: min_AF_ratio_100, min_AF_softclip_ratio_100, min_AF_softclip, min_AF_softclip_ratio_90, and max_alt_count. In some embodiments, the decision trees also receive four or more of the following parameters: min_AF_ratio_100, min_AF_softclip_ratio_100, min_AF_softclip, min_AF_softclip_ratio_90, and max_alt_count. In some embodiments, the decision trees also receive the following parameters: min_AF_ratio_100, min_AF_softclip_ratio_100, min_AF_softclip, min_AF_softclip_ratio_90, and max_alt_count. In some embodiments, the decision trees also receive min_AF_softclip_ratio_100. In some embodiments, the decision trees also receive min_AF_ratio_100 and min_AF_softclip_ratio_10. In some embodiments, the decision trees also receive min_AF_ratio_100, min_AF_softclip_ratio_100, and min_AF_softclip. In some embodiments, the decision trees also receive min_AF_ratio_100, min_AF_softclip_ratio_100, min_AF_softclip, and min_AF_softclip_ratio_90.

In some embodiments, the decision trees also receive 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, or all of the following parameters: max_AF_ratio_100, AF, frac_amps_with_evidence, pan_tumor_prior, amplicon_variant_count, AF_frac_pos_max, analysis_variant_count, min_depth, min_AF, pct_noisy_positions, avg_cov_rel_chip, max_AF_ratio_99, AF_softclip, pan_tumor_prior_gene, min_AF_ratio_99, within_tumor_prior_gene, max_AF_ratio_50, min_AF_softclip_ratio_50, max_AF_ratio_90, depth, num_covering_amps, DNA_avg_coverage, max_AF_softclip, frac_max_af, max_depth, max_AF_softclip_ratio_99, min_AF_ratio_90, max_AF, max_AF_softclip_ratio_100, max_AF_softclip_ratio_90, max_softclip_count, best_strand_bias, max_AF_softclip_ratio_50, and min_softclip_count. In some embodiments, the decision trees also receive one or more of the following parameters: max_AF_ratio_100, AF, frac_amps_with_evidence, pan_tumor_prior, amplicon_variant_count, AF_frac_pos_max, analysis_variant_count, min_depth, min_AF, pct_noisy_positions, avg_cov_rel_chip, max_AF_ratio_99, AF_softclip, pan_tumor_prior_gene, min_AF_ratio_99, within_tumor_prior_gene, max_AF_ratio_50, min_AF_softclip_ratio_50, max_AF_ratio_90, depth, num_covering_amps, DNA_avg_coverage, max_AF_softclip, frac_max_af, max_depth, max_AF_softclip_ratio_99, min_AF_ratio_90, max_AF, max_AF_softclip_ratio_100, max_AF_softclip_ratio_90, max_softclip_count, best_strand_bias, max_AF_softclip_ratio_50, and min_softclip_count.

The machine learning classifier is not limited and may be any suitable machine learning classifier type. Machine learning approaches including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach.

In some embodiments, the machine learning classifier is selected from the group consisting of: a neural network, Bayesian classifier, logistic regression, decision tree, gradient-boosted tree, multilayer perceptron, one-vs-rest, and Naive Bayes.

In some embodiments, the machine learning approach is a supervised learning approach. In some embodiments, evaluating the candidate variant with machine learning further comprises evaluating the candidate variant using a random forest classifier.

In some embodiments, the plurality of decision trees further comprises at least one thousand decision trees. In some embodiments, the plurality of decision trees further comprises at least ten thousand decision trees. In some embodiments, the plurality of decision trees further comprises at least fifty thousand decision trees.

In some embodiments, the plurality of decision trees further comprises a plurality of decision trees for each mutation. In some embodiments, the plurality of decision trees for each mutation comprises at least 5 decision trees per each mutant. In some embodiments, the plurality of decision trees for each mutation comprises at least 50 decision trees per each mutant. In some embodiments, the plurality of decision trees for each mutation comprises at least 100 decision trees per each mutant. In some embodiments, the plurality of decision trees for each mutation comprises at least 500 decision trees per each mutant. In some embodiments, the plurality of decision trees for each mutation comprises at least 1000 decision trees per each mutant. In some embodiments, the plurality of decision trees for each mutation comprises at least 5000 decision trees per each mutant.

Some embodiments of the methods disclosed herein further comprise training the machine learning classifier using a training data set of sequences that include identified mutations. In some embodiments, the training data set of sequences was obtained in part from low quality biological samples (e.g., at least 1%, at least 2%, at least 3%, at least 5%, at least 7.5%, at least 10% are low quality biological samples). In some embodiments, the mutations are identified via expert review. In some embodiments, the training data set of sequences was obtained in part from low quality biological samples (e.g., at least 1%, at least 2%, at least 3%, at least 5%, at least 7.5%, at least 10% is low quality) and the mutations are identified via expert review.

In some embodiments, the training the machine learning classifier further comprises optimizing parameters of the machine learning classifier until the machine learning classifier produces output describing the mutations identified in the training set (e.g., the mutations identified by expert review).

In some embodiments, optimizing parameters of the machine learning classifier further comprises selecting a plurality of feature categories. In some embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, or 44 feature categories (e.g., the feature categories provided in Table 2, 3 or 4 below. In some embodiments, the feature categories min_AF_ratio_50, min_alt_count, min_AF_softclip_ratio_99, count, within_tumor_prior, min_AF_ratio_100, min_AF_softclip_ratio_100, min_AF_softclip, min_AF_softclip_ratio_90, max_alt_count, max_AF_ratio_100, AF, frac_amps_with_evidence, pan_tumor_prior, amplicon_variant_count, AF_frac_pos_max, analysis_variant_count, min_depth, min_AF, pct_noisy_positions, avg_cov_rel_chip, max_AF_ratio_99, AF_softclip, pan_tumor_prior_gene, min_AF_ratio_99, within_tumor_prior_gene, max_AF_ratio_50, min_AF_softclip_ratio_50, max_AF_ratio_90, depth, num_covering_amps, DNA_avg_coverage, max_AF_softclip, frac_max_af, max_depth, max_AF_softclip_ratio_99, min_AF_ratio_90, max_AF, max_AF_softclip_ratio_100, max_AF_softclip_ratio_90, max_softclip_count, best_strand_bias, max_AF_softclip_ratio_50, and min_softclip_count. In some embodiments, the features are the first 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, or 44 provided in Table 3 from the top of the table.

In some embodiments, the method further comprises generating, using the computer, an overall confidence score for the candidate variant being a real mutation based on the classifications of all of the plurality of decision trees. In some embodiments, the overall confidence score is on a scale of 0-1, with a 1 being a 100% confidence for a real mutation. In some embodiments, a candidate variant is called as a real mutation when the confidence score is greater than 0.3, 0.4, 0.5, 0.6, 0.7, 0.75, 0.8, 0.85, 0.9, 0.95, 0.98, or 0.99. In some embodiments, wherein the candidate variant is detected in a high quality sample, the candidate variant is called as a real mutation when the confidence score is greater than 0.3. In some embodiments, wherein the candidate variant is detected in a high low sample, the candidate variant is called as a real mutation when the confidence score is greater than 0.7. In some embodiments, a candidate variant is called as a real mutation when the confidence score is greater than 0.9.

In some embodiments, one or more of the decision trees receive read coverage parameter. In some embodiments, one or more of the decision trees receive parameters are selected from min_AF_ratio_50; min_alt_count; min_AF_softclip_ratio_99; count; min_AF_softclip; min_AF_softclip_ratio_100; within_tumor_prior; min_AF_ratio_100; max_AF_ratio_100; and max_alt_count.

In some embodiments wherein the training data set comprises a plurality of known single-nucleotide variants (SNVs) and Indels, the method comprising: detecting at least one SNV or Indel in the nucleic acid; validating the detected SNV or Indel as present in the nucleic acid using the classification model; and providing a report that describes the nucleic as including the SNV or Indel.

In some embodiments wherein the training data set comprises a plurality of known single-nucleotide variants (SNVs), the method comprising: detecting at least one SNV in the nucleic acid; validating the detected SNV as present in the nucleic acid using the classification model; and providing a report that describes the nucleic as including the SNV.

In some embodiments wherein the training data set comprises a plurality of insertions and/or deletions (Indel), the method comprising: detecting at least one Indel in the nucleic acid; validating the detected indel as present in the nucleic acid using the classification model; and providing a report that describes the nucleic as including the indel.

Some aspects of the present disclosure are directed to a system for identifying mutations from a patient sample, comprising a computer system having a processor, memory and a plurality of lines of instructions; a machine learning classifier executed by the processor of the computer system, the machine learning classifier being configured to: evaluate a candidate variant against the plurality of decision trees trained to detect mutations in the candidate variant, wherein each decision tree classifies the candidate variant as at least one of present or not present; and classify the candidate variant as present or not present based on classifications of each of the plurality of decision trees.

The machine learning classifier is not limited and may be any suitable machine learning methodology described herein. In some embodiments, the machine learning classifier is selected from the group consisting of: a neural network, Bayesian classifier, logistic regression, decision tree, gradient-boosted tree, multilayer perceptron, one-vs-rest, and Naive Bayes. In some embodiments, the machine learning classifier is further configured to evaluate the candidate variant using a random forest classifier. The number of decision trees is not limited and may be any suitable number described herein. In some embodiments, the plurality of decision trees further comprises at least one thousand decision trees. In some embodiments, the plurality of decision trees further comprises a plurality of decision trees for each mutation.

In some embodiments, the processor is further configured to train the machine learning classifier using a training data set of sequences that include known mutations. In some embodiments, the training data set of sequences was obtained in part from low quality biological samples and wherein the mutations are identified via expert review.

As used herein, low quality biological samples are samples comprising significantly degraded nucleotide sequences (e.g., DNA). In some embodiments, the low quality biological samples are from samples that have undergone formalin fixation and paraffin embedding (FFPE). In some embodiments, the low quality biological samples comprise at least 2-fold, 5-fold, 10-fold, or more chemically modified nucleotides or chemical crosslinks than a freshly obtained and untreated biological sample. In some embodiments, the low quality biological samples comprise at least 2-fold, 5-fold, 10-fold, or more degraded sequences (DNA sequences) than a freshly obtained and untreated biological sample.

In some embodiments, the processor is further configured to optimize parameters of the machine learning classifier until the machine learning classifier produces output describing the known mutations.

In some embodiments, the processor is further configured to select a plurality of feature categories. The feature categories are not limited and may be any feature categories described herein. In some embodiments, the feature categories are provided in Table 2, 3, or 4. In some embodiments, the feature categories are provided in Table 3.

In some embodiments, the processor is further configured to generate an overall confidence score for the candidate variant being a real mutation based on the classifications of all of the plurality of decision trees.

In embodiments of the methods and systems disclosed herein, the biological sample (i.e., sample) is any suitable sample type. In some embodiments, the sample is from plasma, blood, serum, saliva, sputum, stool, a tumor, cell free DNA, circulating tumor cell, or other biological sample. In some embodiments, the sample is a blood sample. In some embodiments, the biological sample is a tumor specimen. In some embodiments, the sample is from a subject having or at risk of having cancer. The type of cancer is not limited and may be any suitable cancer. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is lung or prostate cancer.

In some embodiments, the cancer is selected from adrenal, biliary, bladder, brain, breast, cervical, colon and rectum, endometrium, esophagus, head and neck, kidney, liver, lung-NSCLC, lung—Other, lymphoma, melanoma, meninges, NSCLC, non-melanoma skin, ovary, pancreas, prostate, sarcoma, small intestine, stomach, thymus, or thyroid cancer. In some embodiments, the cancer is selected from lung, bladder, colon, gastric, head and neck, breast, prostate, non-small cell lung adenocarcinoma, non-small cell lung squamous cell carcinoma, bladder urothelial carcinoma, colorectal, brain or pancreatic cancer.

In some embodiments, the biological sample comprises less genomic material than prior mutant calling methods permit. In some embodiments, the biological sample comprises less than about 25 nanograms (ng) of genomic material. In some embodiments, the biological sample comprises less than about 20 ng of genomic material. In some embodiments, the biological sample comprises less than about 15 ng of genomic material. In some embodiments, the biological sample comprises less than about 12 ng of genomic material. In some embodiments, the biological sample comprises less than 10 ng of genomic material. In some embodiments, the biological sample comprises less than 7.5 ng of genomic material. In some embodiments, the biological sample comprises less than 5 ng of genomic material.

In some embodiments, the biological sample has undergone fixation. The method of fixation is not limited and may be any method of fixation known in the art. In some embodiments, fixation includes formalin fixation which is known in the art to result in an abundance of C>T mutations thought to be due to deamination.

The area of the biological sample (mm²) is not limited. In some embodiments, the area of the biological sample is less than the area needed for variant calling methods used in the art. In some embodiments, the biological sample is a sample having an area of less than 5 mm². In some embodiments, the biological sample is a sample having an area of less than 10 mm². In some embodiments, the biological sample is a sample having an area of less than 15 mm². In some embodiments, the biological sample is a sample having an area of less than 20 mm². In some embodiments, the biological sample is a sample having an area of less than 25 mm². In some embodiments, the biological sample is a sample having an area of less than 30 mm². In some embodiments, the biological sample is a sample having an area of less than 35 mm². In some embodiments, the biological sample is a sample having an area of less than 40 mm².

The tumor content of the biological sample is not limited. In some embodiments, the tumor content of the biological sample is less than the tumor content required in methods of variant calling practiced in the art. In some embodiments, the biological sample is a sample having a tumor content of less than 40%. In some embodiments, the biological sample is a sample having a tumor content of less than 30%. In some embodiments, the biological sample is a sample having a tumor content of less than 20%. In some embodiments, the biological sample is a sample having a tumor content of less than 17%. In some embodiments, the biological sample is a sample having a tumor content of less than 15%. In some embodiments, the biological sample is a sample having a tumor content of less than 12%. In some embodiments, the biological sample is a sample having a tumor content of less than 10%.

Methods of determining or calculating tumor content are not limited and may be any suitable method known in the art.

Methods of preparing biological specimens and sequencing data sets are not limited and may be any suitable method used in the art. In some embodiments, the method comprises one or more steps comprising 1) receiving a sample (FFPE block or slides); 2) reviewing H&E stained slide for tumor content; 3) Cutting additional slides as necessary; 4) Scraping cells from slides to into tubes (performing macrodissection if indicated by pathologist); 5) Extracting nucleic acid (fully automated batch process); 6) Preparing DNA libraries (largely automated batch process); 7) Loading DNA libraries to sequencing chip (fully automated); 8) Sequencing chips via NGS (fully automated); and 9) Data analysis.

In some embodiments, the biological sample has been stored for at least about 1 year prior to sequencing. In some embodiments, the biological sample has been stored for at least about 2 years prior to sequencing. In some embodiments, the biological sample has been stored for at least about 3 years prior to sequencing. In some embodiments, the biological sample has been stored for at least about 4 years prior to sequencing. In some embodiments, the biological sample has been stored for at least about 5 years prior to sequencing. In some embodiments, the biological sample has been stored for at least about 10 years prior to sequencing.

In some embodiments, the mutation sequencing data set is low quality. In some embodiments, the mutation sequencing data set comprises less than 1000 reads of the candidate variant of interest. In some embodiments, the mutation sequencing data set comprises less than 600 reads of the candidate variant of interest. In some embodiments, the mutation sequencing data set comprises less than 500 reads of the candidate variant of interest. In some embodiments, the mutation sequencing data set comprises less than 400 reads of the candidate variant of interest. In some embodiments, the mutation sequencing data set comprises less than 300 reads of the candidate variant of interest. In some embodiments, the mutation sequencing data set has been obtained in a batch with a plurality of other sequencing data sets and comprises less than 50% of the average number of reads in the plurality of other sequencing data sets. In some embodiments, no more than 1% of the positions of the sequencing data set have more that 3% of non-reference alignments.

In some embodiments the determination of the mutation or mutations in the sample leads to and is followed by particular treatment steps intended to be therapeutic for the identified mutation(s), mutation symptomology, and/or disorder associated with the mutation(s). In such embodiments the treatment steps can be treatment with a suitable agent or agents, including combination treatment regimens. Suitable treatment steps can include, for example, administration of appropriate gene therapy agents, such as TALENs, zinc fingers and/or Crispr agents and suitable guide sequences, for correcting the mutation(s) in the subject from whom the sample was obtained. Suitable treatment steps can also include chemotherapeutic agents and regimens which are efficacious in treatment of cancers associated with identified mutation(s). The particular treatment will be determined based upon the mutation(s) identified using the methods described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### Examples

### Training Set

Model parameters were trained using a corpus of paired raw sequencing data and expert review data from comprehensive genomic profiling tests for a consecutive cohort of 3,020 clinical samples spanning 30 tumor types [Adrenal, Biliary, Bladder, Brain, Breast, Cervical, Colon and Rectum, Endometrium, Esophagus, Head and Neck, Kidney, Liver, Lung-NSCLC, Lung—Other, Lymphoma, Melanoma, Meninges, NSCLC, Non-Melanoma Skin, other, Ovary, Pancreas, Prostate, Sarcoma, Small Intestine, Stomach, Thymus, Thyroid, and unknown primary]. Expert review included raw data review and reporting decision arbitration for individual mutations; this review was guided by clinical context, overall sequencing data quality (per sample and per batch), and orthogonal testing results. Notably, the test includes several hundred kilobases of genomic content with redundant coverage generated by two independent library preparations; discordances within these redundant regions were manually reviewed and adjudicated as false positive or false negative based on all supporting evidence. Model training was performed using default parameters as described in the open-source XGBoost library (version 1.2.1). No low-quality data were excluded from training or subsequent performance evaluation; instead, raw quality control metrics were provided as input to the classifier as additional signal for contextualizing the potential significance of individual mutation candidate specific inputs. The feature weights obtained from model training are provided in Table 3 below.

### Prospective Validation

After model training, performance was validated prospectively in a separate cohort of 3,437 consecutive clinical samples. The results are shown in Table 1. Sensitivity and positive predictive value were evaluated in comparison to the clinically reported results following manual expert review. Performance was evaluated in the following subgroups:
Mutation type:
single nucleotide variant (SNV)
insertion/deletion (INDEL)
Clinical significance category:
Companion diagnostic markers (CDx)-mutations with existing clinical indications, such as BRAF p. V600E, which may indicate vemurafenib in melanomas
Hotspot-specific mutations with known functional, clinical and/or recurrence evidence supporting their inclusion in clinical reporting
De novo-non-hotspot mutations with clear functional impact via truncated or significantly altered downstream gene transcription
Variants of Unknown Significance (VUS)-non-hotspot mutations with unknown functional impact and significance

TABLE 1

| group | type | n | sens. | PPV |
|---|---|---|---|---|
| CDx | INDEL | 94 | 95.74% | 95.16% |
| CDx | SNV | 669 | 99.24% | 95.21% |
| VUS | INDEL | 692 | 82.29% | 92.77% |
| VUS | SNV | 16,794 | 90.44% | 85.15% |
| de novo | INDEL | 1,196 | 94.34% | 90.31% |
| de novo | SNV | 1,923 | 97.26% | 91.79% |
| hotspot | INDEL | 164 | 94.19% | 97.76% |
| hotspot | SNV | 3,738 | 97.67% | 93.34% |

### Example Calculations

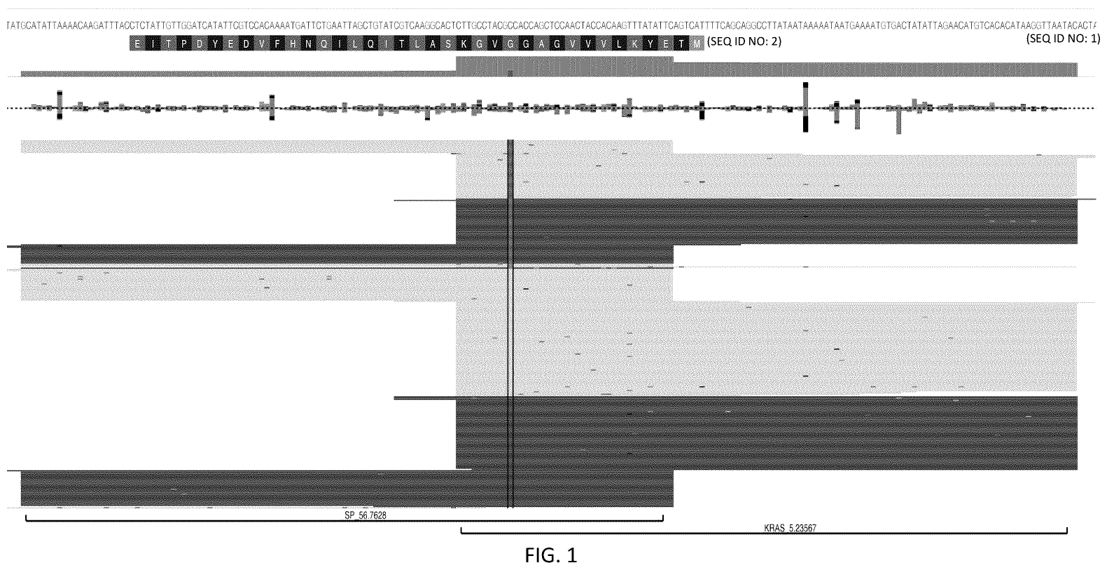

### Positive Example-KRAS Chr12:25398280 C>T (FIG. 1)

Several factors support the significance of this KRAS p.G13D mutation, which yielded an overall score of 0.9998 (range 0-1)—see details in table below:

The mutation is covered by two independent amplicons, both of which support the mutation:
num_covering_amps=2
frac_amps_with_evidence=1

The read evidence is well above expected background error for that specific nucleotide change
min_AF_ratio_100=73.261

The read variant allele frequency is similar on both strands (range 0-1):
best_strand_bias=0.919

Both pan tumor and within tumor prior probabilities are elevated for both this specific mutation and gene:
within_tumor_prior=0.078
within_tumor_prior_gene=0.488

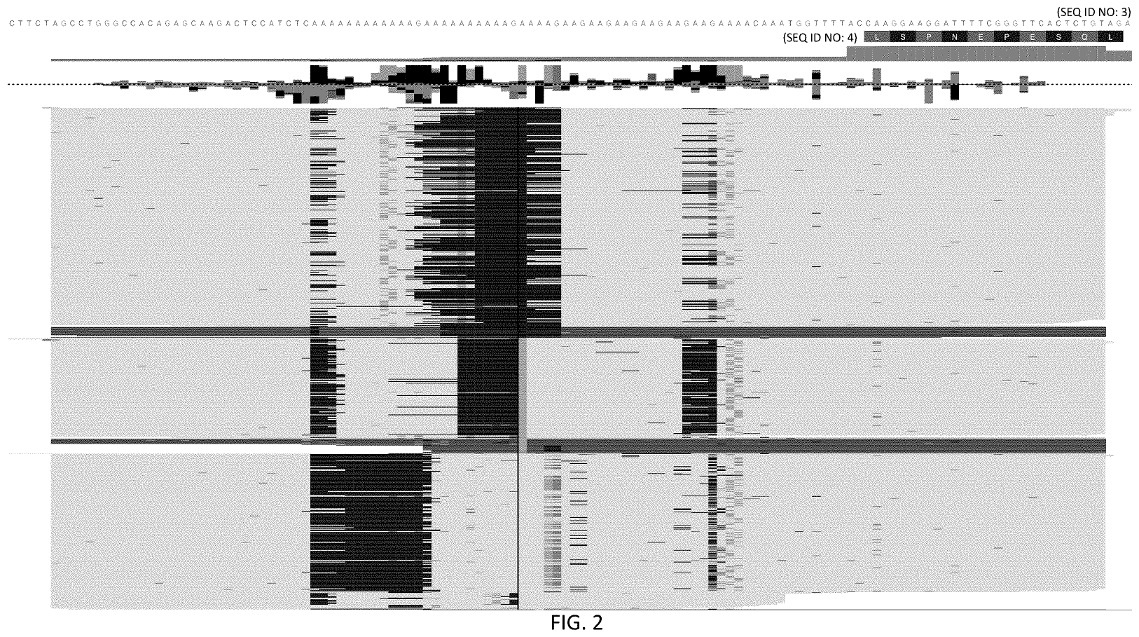

### Negative Example 1-BRCA1 Chr17:41256098 A>G (FIG. 2)

Although the raw variant allele frequency (AF) of 14.6% is relatively high, several factors argue against this mutation candidate:

Allele frequencies for both the forward and reverse strands are less than the expected background:
max_AF_ratio_100=0.711

The read support is highly strand-biased (variant allele frequencies are different on each strand):
best_strand_bias=0.102

There are many mutation candidates of the same nucleotide change (A>G) present on the same amplicon:
amplicon_variant_count=13

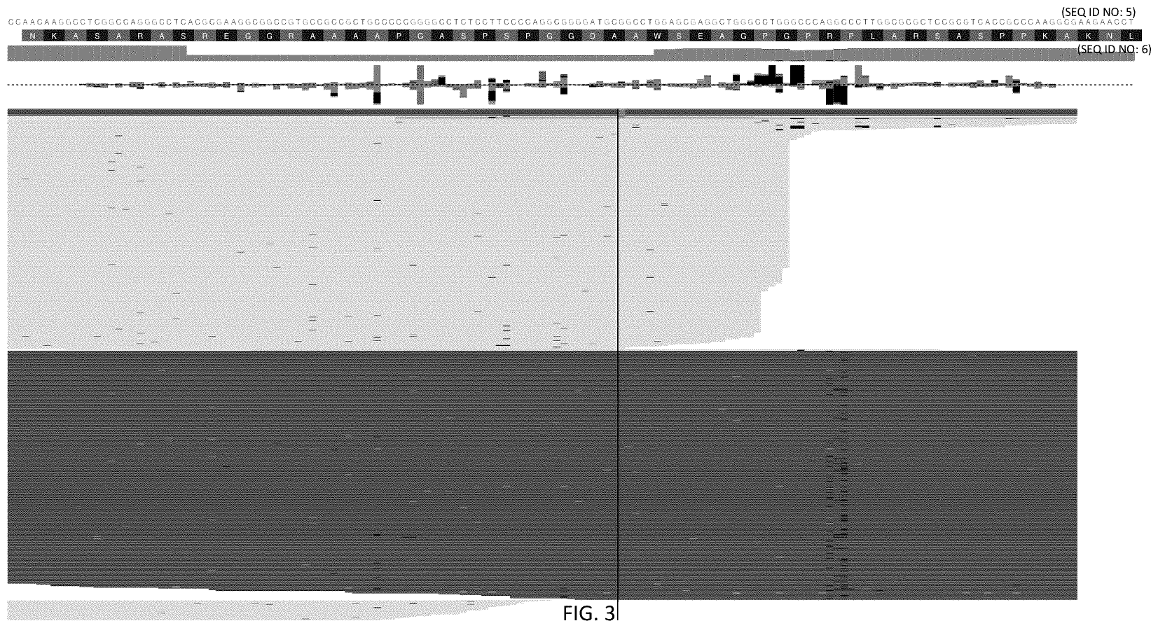

### Negative Example 2: MSH6 Chr2:48010515 G>a (FIG. 3)

This candidate is typical of sequencing errors that are often present at variant allele frequencies high enough to trigger false positive results in systems that do not consider second-order evidence such as strand bias, multiple amplicon coverage, and prior probabilities. Though the local sequencing data quality is high (nearly all bases are colored pink or purple, indicating match to the expected reference sequence), and one strand is well above the expected background error (max_AF_ratio_100=5.262), there is no supporting evidence on the reverse strand reads—this manifests in several different parameters:
best_strand_bias=0
min_AF=0
min_alt_count=0

Variant candidates like this with marginal read support can be real—for example, it is common to have biased read coverage such that only one strand has sufficient reads to support analysis; in such cases where read support is not definitive, prior probabilities and multiple amplicon coverage can help resolve such ambiguity. In this case, neither parameter lends support:
num_covering_amps=1
pan_tumor_prior, pan_tumor_prior_gene, within_tumor_prior, within_tumor_prior_gene <0.01

The results for positive example 1 and negative examples 1-2 are shown in Table 2:

| gene | KRAS | BRCA1 | MSH6 |
|---|---|---|---|
| score | 0.99984086 | 0.00000005 | 0.00010444 |
| AF | 0.398 | 0.146 | 0.029 |
| AF_frac_pos_max | 1.000 | 1.000 | 1.000 |
| AF_softclip | 0.398 | 0.146 | 0.029 |
| DNA_avg_coverage | 3545 | 3545 | 3545 |
| amplicon_variant_count | 1 | 13 | 2 |
| analysis_variant_count | 0.003 | 0.002 | 0.003 |
| avg_cov_rel_chip | 1.213 | 1.213 | 1.213 |
| best_strand_bias | 0.919 | 0.102 | 0.000 |
| count | 834 | 132 | 9 |
| depth | 2096 | 903 | 308 |
| frac_amps_with_evidence | 1.000 | 1.000 | 1.000 |
| frac_max_af | 0.605 | 0.222 | 0.044 |
| max_AF | 0.415 | 0.215 | 0.030 |
| max_AF_ratio_100 | 86.363 | 0.711 | 5.262 |
| max_AF_ratio_50 | 414542.021 | 5.943 | 29900.332 |
| max_AF_ratio_90 | 274.028 | 1.664 | 18.623 |
| max_AF_ratio_99 | 107.310 | 0.791 | 6.129 |
| max_AF_softclip | 0.415 | 0.215 | 0.030 |
| max_AF_softclip_ratio_100 | 86.363 | 0.711 | 5.262 |
| max_AF_softclip_ratio_50 | 414542.021 | 5.943 | 29900.332 |
| max_AF_softclip_ratio_90 | 274.028 | 1.664 | 18.623 |
| max_AF_softclip_ratio_99 | 107.310 | 0.791 | 6.129 |
| max_alt_count | 439 | 125 | 9 |
| max_depth | 1059 | 582 | 301 |
| max_softclip_count | 0.000 | 0.000 | 0.000 |
| min_AF | 0.381 | 0.022 | 0.000 |
| min_AF_ratio_100 | 73.261 | 0.575 | 0.000 |
| min_AF_ratio_50 | 380906.461 | 2.169 | 0.000 |
| min_AF_ratio_90 | 194.735 | 1.072 | 0.000 |
| min_AF_ratio_99 | 101.184 | 0.736 | 0.000 |
| min_AF_softclip | 0.381 | 0.022 | 0.000 |
| min_AF_softclip_ratio_100 | 73.261 | 0.575 | 0.000 |
| min_AF_softclip_ratio_50 | 380906.461 | 2.169 | 0.000 |
| min_AF_softclip_ratio_90 | 194.735 | 1.072 | 0.000 |
| min_AF_softclip_ratio_99 | 101.184 | 0.736 | 0.000 |
| min_alt_count | 395 | 7 | 0 |
| min_depth | 1037 | 321 | 7 |
| min_softclip_count | 0 | 0 | 0 |
| num_covering_amps | 2 | 1 | 1 |
| pan_tumor_prior | 0.015 | 0.000 | 0.000 |
| pan_tumor_prior_gene | 0.228 | 0.017 | 0.006 |
| pct_noisy_positions | 0.035 | 0.035 | 0.035 |
| within_tumor_prior | 0.078 | 0.000 | 0.000 |
| within_tumor_prior_gene | 0.488 | 0.010 | 0.007 |

TABLE 3
Features with weight

| Feature | Weight |
|---|---|
| min_AF_ratio_50 | 0.516085 |
| min_alt_count | 0.076251 |
| min_AF_softclip_ratio_99 | 0.060297 |
| count | 0.039028 |
| within_tumor_prior | 0.035266 |
| min_AF_ratio_100 | 0.029994 |
| min_AF_softclip_ratio_100 | 0.028688 |
| min_AF_softclip | 0.028060 |
| min_AF_softclip_ratio_90 | 0.015178 |
| max_alt_count | 0.014694 |
| max_AF_ratio_100 | 0.012226 |
| AF | 0.011352 |
| frac_amps_with_evidence | 0.010639 |
| pan_tumor_prior | 0.010220 |
| amplicon_variant_count | 0.008132 |
| AF_frac_pos_max | 0.007748 |
| analysis_variant_count | 0.007694 |
| min_depth | 0.006241 |
| min_AF | 0.006093 |
| pct_noisy_positions | 0.005539 |
| avg_cov_rel_chip | 0.005173 |
| max_AF_ratio_99 | 0.004943 |
| AF_softclip | 0.004925 |
| pan_tumor_prior_gene | 0.004815 |
| min_AF_ratio_99 | 0.004226 |
| within_tumor_prior_gene | 0.004098 |
| max_AF_ratio_50 | 0.003961 |
| min_AF_softclip_ratio_50 | 0.003600 |
| max_AF_ratio_90 | 0.003043 |
| depth | 0.002812 |
| num_covering_amps | 0.002789 |
| DNA_avg_coverage | 0.002763 |
| max_AF_softclip | 0.002637 |
| frac_max_af | 0.002610 |
| max_depth | 0.002566 |
| max_AF_softclip_ratio_99 | 0.002521 |
| min_AF_ratio_90 | 0.002356 |
| max_AF | 0.002139 |
| max_AF_softclip_ratio_100 | 0.002023 |
| max_AF_softclip_ratio_90 | 0.002015 |
| max_softclip_count | 0.001822 |
| best_strand_bias | 0.001468 |
| max_AF_softclip_ratio_50 | 0.001271 |
| min_softclip_count | 0.000000 |

TABLE 4
Feature Key

| Feature | description |
|---|---|
| AF | allele frequency - fraction of all covering reads that support |
| | the variant |
| AF_frac_pos_max | AF as a fraction of the highest observed AF for any variant |
| | candidate at the same genomic coordinate |
| AF_softclip | AF when including reads that truncate alignment at the |
| | variant site as supporting evidence |
| DNA_avg_coverage | Average depth of read coverage across all amplicons |
| amplicon_variant_count | Total number of variant candidates of the same mutation |
| | class (A > C, A > G, A > T, C > G, C > T, C > A, insertion or |
| | deletion) on the same amplicon |
| analysis_variant_count | Total number of variant candidates of the same mutation |
| | class (A > C, A > G, A > T, C > G, C > T, C > A, insertion or |
| | deletion) across all amplicons |
| avg_cov_rel_chip | Average depth of read coverage across all amplicons as a |
| | fraction of the median sample-level value in the same |
| | sequencing batch |
| best_strand_bias | ratio of minimum strand-level AF to maximum strand-level |
| | AF, considering all combinations of full read vs. softclip AF |
| | values |
| Count | total supporting read count |
| Depth | total covering read count |
| frac_amps_with_evidence | fraction of all covering amplicons that support the candidate |
| frac_max_af | AF as a fraction of the highest observed AF in the same |
| | sample (excluding known germline variants) |
| max_AF | max strand-level AF (forward or reverse) |
| max_AF_ratio_100 | max_AF as a fraction of the maximum observed error for the |
| | allele in the background error model |
| max_AF_ratio_50 | max_AF as a fraction of the median observed error for the |
| | allele in the background error model |
| max_AF_ratio_90 | max_AF as a fraction of the 90th pctile of observed errors for |
| | the allele in the background error model |
| max_AF_ratio_99 | max_AF as a fraction of the 99th pctile of observed errors for |
| | the allele in the background error model |
| max_AF_softclip | max strand-level AF (forward or reverse) when considering |
| | truncated reads as supporting evidence (forward or reverse) |
| max_AF_softclip_ratio_100 | max_AF_softclip as a fraction of the maximum observed |
| | error for the allele in the background error model |
| max_AF_softclip_ratio_50 | max_AF_softclip as a fraction of the median observed error |
| | for the allele in the background error model |
| max_AF_softclip_ratio_90 | max_AF_softclip as a fraction of the 90th pctile of observed |
| | errors for the allele in the background error model |
| max_AF_softclip_ratio_99 | max_AF_softclip as a fraction of the 99th pctile of observed |
| | errors for the allele in the background error model |
| max_alt_count | max strand-level alt count (forward or reverse) |
| max_depth | max strand-level coverage depth (forward or reverse) |
| max_softclip_count | max strand-level alt count when considering truncated reads |
| | as supporting evidence (forward or reverse) |
| min_AF | min strand-level AF (forward or reverse) |
| min_AF_ratio_100 | min_AF as a fraction of the maximum observed error for the |
| | allele in the background error model |
| min_AF_ratio_50 | min_AF as a fraction of the median observed error for the |
| | allele in the background error model |
| min_AF_ratio_90 | min_AF as a fraction of the 90th pctile of observed errors for |
| | the allele in the background error model |
| min_AF_ratio_99 | min_AF as a fraction of the 99th pctile of observed errors for |
| | the allele in the background error model |
| min_AF_softclip | min strand-level AF (forward or reverse) when considering |
| | truncated reads as supporting evidence (forward or reverse) |
| min_AF_softclip_ratio_100 | min_AF_softclip as a fraction of the maximum observed |
| | error for the allele in the background error model |
| min_AF_softclip_ratio_50 | min_AF_softclip as a fraction of the median observed error |
| | for the allele in the background error model |
| min_AF_softclip_ratio_90 | min_AF_softclip as a fraction of the 90th pctile of observed |
| | errors for the allele in the background error model |
| min_AF_softclip_ratio_99 | min_AF_softclip as a fraction of the 99th pctile of observed |
| | errors for the allele in the background error model |
| min_alt_count | min strand-level alt count (forward or reverse) |
| min_depth | min strand-level coverage depth (forward or reverse) |
| min_softclip_count | min strand-level alt count when considering truncated reads |
| | as supporting evidence (forward or reverse) |
| num_covering_amps | number of amplicons whose coordinate bounds fully cover |
| | the variant candidate |
| pct_noisy_positions | fraction of positions on the panel with >3% non-reference |
| | support |
| pan_tumor_prior | population frequency of variant observed in pan-tumor cohort |
| pan_tumor_prior_gene | population frequency of variants within the same gene |
| | observed in pan-tumor cohort |
| within_tumor_prior | population frequency of variant observed in within-tumor |
| | cohort |
| within_tumor_prior_gene | population frequency of variants within the same gene |
| | observed in within-tumor cohort |

What is claimed is:

1. A method for identifying mutations from a patient sample, comprising: evaluating, using a computer having a machine learning classifier, a candidate variant against a plurality of decision trees trained to detect mutations in the candidate variant with a gradient boosting algorithm, wherein each decision tree classifies the candidate variant as at least one of present or not present; and
classifying, using the computer, the candidate variant as present or not present based on classifications of each of the plurality of decision trees,
wherein the decision trees receive the following parameters:
min_AF_ratio_50; min_alt_count; min_AF_softclip_ratio_99; count; and
within_tumor_prior.

2. The method of claim 1, wherein the decision trees also receive the following parameters:
min_AF_ratio_100, min_AF_softclip_ratio_100, min_AF_softclip,
min_AF_softclip_ratio_90, and max_alt_count.

3. The method of any one of claims 1-2, wherein the decision trees also receive one or more of the following parameters:
max_AF_ratio_100, AF, frac_amps_with_evidence, pan_tumor_prior,
amplicon_variant_count, AF_frac_pos_max, analysis_variant_count, min_depth,
min_AF, pct_noisy_positions, avg_cov_rel_chip, max_AF_ratio_99, AF_softclip,
pan_tumor_prior_gene, min_AF_ratio_99, within_tumor_prior_gene,
max_AF_ratio_50, min_AF_softclip_ratio_50, max_AF_ratio_90, depth,
num_covering_amps, DNA_avg_coverage, max_AF_softclip, frac_max_af,
max_depth, max_AF_softclip_ratio_99, min_AF_ratio_90, max_AF,
max_AF_softclip_ratio_100, max_AF_softclip_ratio_90, max_softclip_count,
best_strand_bias, max_AF_softclip_ratio_50, and min_softclip_count.

4. The method of any one of claims 1-3, wherein evaluating the candidate variant further comprises evaluating the candidate variant using a random forest classifier.

5. The method of claim 4, wherein the plurality of decision trees further comprises at least one thousand decision trees.

6. The method of claim 4, wherein the plurality of decision trees further comprises a plurality of decision trees for each mutation.

7. The method of any one of claims 1-3, further comprising training the machine learning classifier using a training data set of sequences that include identified mutations.

8. The method of claim 7, wherein the training data set of sequences was obtained in part from low quality biological samples and wherein the mutations are identified via expert review.

9. The method of any one of claims 7-8, wherein training the machine learning classifier further comprises optimizing parameters of the machine learning classifier until the machine learning classifier produces output describing the known mutations.

10. The method of claim 9, wherein optimizing parameters of the machine learning classifier further comprises selecting a plurality of feature categories.

11. The method of any one of claims 1-3, wherein the machine learning classifier is selected from the group consisting of: a neural network, Bayesian classifier, logistic regression, decision tree, gradient-boosted tree, multilayer perceptron, one-vs-rest, and Naive Bayes.

12. The method of any one of claims 1-3, further comprising generating, using the computer, an overall confidence score for the candidate variant being a real mutation based on the classifications of all of the plurality of decision trees.

13. The method of any one of claims 1-3, further comprising providing a report that describes the candidate variant as including the mutation or structural alteration.

14. The method of claim 13, wherein describing the structural alteration comprises: comparing the sequence reads to a reference to detect an indicium of the structural alteration; and validating the structural alteration as present in the nucleic acid using the classification model.

15. The method of any one of claims 1-14, wherein one or more of the decision trees receive parameters selected from the group consisting of: sample type; FASTQ quality score; alignment score; read coverage; and an estimated probability of error.

16. The method of any one of claims 7-10, wherein the training data set comprises a plurality of known single-nucleotide variants (SNVs) and insertions/deletions (Indels), the method comprising: detecting at least one SNV or Indel in the nucleic acid; validating the detected SNV or Indel as present in the nucleic acid using the classification model; and providing a report that describes the nucleic as including the SNV or Indel.

17. A system for identifying mutations from a patient sample, comprising:
a computer system having a processor, memory and a plurality of lines of instructions;
a machine learning classifier executed by the processor of the computer system, the machine learning classifier being configured to: evaluate a candidate variant against the plurality of decision trees trained to detect mutations in the candidate variant,
wherein each decision tree classifies the candidate variant as at least one of present or not present; and
classify the candidate variant as a real mutation based on classifications of each of the plurality of decision trees.

18. The system of claim 17, wherein machine learning classifier is further configured to evaluate the candidate variant using a random forest classifier.

19. The system of any one of claims 17-18, wherein the plurality of decision trees further comprises at least one thousand decision trees.

20. The system of any one of claims 17-19, wherein the plurality of decision trees further comprises a plurality of decision trees for each mutation.

21. The system of any one of claims 17-20, wherein the processor is further configured to train the machine learning classifier using a training data set of sequences that include known mutations.

22. The system of claim 21, wherein the training data set of sequences was obtained in part from low quality biological samples and wherein the mutations are identified via expert review.

23. The system of any one of claims 17-22, wherein the processor is further configured to optimize parameters of the machine learning classifier until the machine learning classifier produces output describing the known mutations.

24. The system of any one of claims 17-23, wherein the processor is further configured to select a plurality of feature categories.

25. The system of claim 17, wherein the machine learning classifier is selected from the group consisting of: a neural network, Bayesian classifier, logistic regression, decision tree, gradient-boosted tree, multilayer perceptron, one-vs-rest, and Naive Bayes.

26. The system of any one of claims 17-25, wherein the processor is further configured to generate an overall confidence score for the candidate variant being a real mutation based on the classifications of all of the plurality of decision trees.

27. The method of claim 1 or the system of claim 17, wherein the sample is from plasma, blood, serum, saliva, sputum, stool, a tumor, cell free DNA, circulating tumor cell, or other biological sample.

28. The method or system of claim 27, wherein the sample is from a subject having or at risk of having cancer.

29. The method or system of claim 28, wherein the cancer is selected from lung, bladder, colon, gastric, head and neck, breast, prostate, non-small cell lung adenocarcinoma, non-small cell lung squamous cell carcinoma, bladder urothelial carcinoma, colorectal, brain or pancreatic cancer.
